(19) United States
(12) Patent Application Publication
(10) Pub. No.: US 2013/0130305 A1
(43) Pub. Date: May 23, 2013

(54) INDENTIFYING ANTIFUNGAL AGENTS THAT INHIBIT IAA OR A YAP FAMILY

(71) Applicant: Reeta Prusty Rao, Newtown, MA (US)

(72) Inventor: Reeta Prusty Rao, Newtown, MA (US)

(21) Appl. No.: 13/697,697

(22) PCT Filed: May 17, 2011
(86) PCT No.: PCT/US2011/036753

§ 371 Date: Feb. 5, 2013

Related U.S. Application Data
(60) Provisional application No. 61/345,320, filed on May 17, 2010.

(51) Int. Cl. C12Q 1/18

(52) U.S. Cl. CPC C12Q 1/18
USPC 435/32

(57) ABSTRACT

The present invention relates to methods of screening for antifungal agents by identifying agents that bind to or otherwise inhibit indole-3-acetic acid (IAA) or that bind to or otherwise inhibit the expression or activity of a protein within the Yap family or a gene encoding a protein within the Yap family.

### CROSS-REFERENCE TO RELATED APPLICATIONS

This application claims the benefit of the filing date of U.S. Application No. 61/345,320, filed May 17, 2010. For the purpose of any U.S. patent that may issue from the present application, the entire content of U.S. Application No. 61/345,320 is hereby incorporated by reference herein.

### STATEMENT REGARDING FEDERALLY FUNDED RESEARCH

This work was supported by a grant from the National Science Foundation under grant number MCB 0517420. The U.S. government has rights in the invention.

### FIELD OF THE INVENTION

The present invention relates to methods of screening for antifungal agents by identifying agents that bind to or otherwise inhibit indole-3-acetic acid (IAA) or a protein within the Yap family.

### BACKGROUND

Currently, fungal infections are treated with agents such as amphotericin B, 5-flucytosin, caspofungin, or various azole derivatives (e.g., fluconazole, ketoconazole, and itraconazole). None of these agents are effective on all fungal infections, and all of the known therapies carry with them some level of toxicity. Increasing levels of resistance to known therapies also presents a problem. Despite efforts to minimize toxicity and maximize potency of the azoles since the 1970s, amphotericin B, a drug introduced in the 1950s, remains the best choice for many serious mycoses, and, in particular, disseminated mycoses. This remains the case despite the drug's significant toxicity and problems with resistance and non-availability of an absorbable oral form for long-term maintenance. Attempts to encapsulate amphotericin B into liposome vesicles to diminish toxicity have proven only moderately successful.

### SUMMARY

The present invention encompasses methods of screening for antifungal agents. The methods can be carried out by steps including: (a) providing an agent; (b) bringing the agent into contact with a protein of the YAP family or a gene encoding a protein of the YAP family; and (c) determining whether the agent inhibits an activity of the protein or the expression of the gene. As discussed further below, inhibition of an activity of the protein indicates that the agent is a putative antifungal agent.

As discussed further below, essentially any type of agent can be screened. For example, the agent provided can be a small molecule (e.g., a small organic compound), a polypeptide, or a nucleic acid. We tend to use the term “polypeptide” to refer to polymers of amino acid residues, regardless of their length, their origin, or other characteristics (e.g., the extent of glycosylation or phosphorylation). The polypeptide can have the sequence of a full-length, naturally-occurring protein, and where the agent tested is a full-length, naturally-occurring protein, it may be one that has been isolated from its source (e.g., a plant or other biological material) or one that is recombinantly produced. Agents that are non-naturally-occurring can also be screened, and these agents can be fragments or other variants of a naturally-occurring polypeptide (e.g., substitution or addition mutants of a naturally-occurring protein). Although there are advantages to using a full-length, naturally-occurring polypeptide in the Yap family (e.g., Yap1 or Cap1) as the target for the putative inhibitors, the screening methods can also be carried out with fragments or other variants of a Yap family member if desired. For example, the screening methods can be carried out with a biologically active fragment of a Yap family member or another biologically active variant thereof (e.g., an addition or substitution mutant expressed, for example, in a yeast cell or other cell type).

Proteins in the Yap family include AaAP1, Afyap1, CgAP1, ChAP1, Yap1, Yap2, PpYap1, Pap1, Cap1, ApyapA, and Nap1, and the present screening methods can be carried out by exposing one or more of these proteins to one or more agents as described herein.

Where the agent is a nucleic acid, it may be either a DNA or RNA molecule, and it may be either naturally or non-naturally-occurring. The nucleic acid can be designed as an antisense oligonucleotide or as a mediator of RNAi. For example, the nucleic acid can have a sequence that inhibits the expression of a gene encoding a protein in the Yap family.

Bringing the agent into contact with the protein can be carried out in a variety of ways. For example, the agent may be brought into contact with a fungal cell that expresses the protein (i.e., a protein of the Yap family). If necessary to increase cellular uptake, the agent may be delivered with a permeation enhancer (e.g., a liposome). Cellular lysates or homogenates can also be used, and the cells, cell lysates or homogenates may be of essentially pure populations of a single cell type or mixed populations of more than one cell type. In some embodiments, the putative anti-fungal agents can be tested on fungi that reside on or within a tissue (e.g., a human tissue such as skin or a human membrane, as is found in the reproductive or respiratory systems). The tissue can be within a test subject, such as a mammal (e.g., a rodent, rabbit, guinea pig, non-human primate, or human).

The fungal cell can be a cell of the genus Alternaria, Aspergillus, Candida, Cochiobolus, Paracoccidioides, Pichia, Schizosaccharomyces, Saccharomyces, Ustilago, Neurospora, or Magnaporthe. As noted, while the methods can be carried out using a single cell type, the screen can also be configured so that multiple cell types are screened together, either by virtue of a mixed culture of cells or by inclusion of different cell types in spatially constrained areas. For example, the screen can be configured on a multiple-well tissue culture plate, with different cell types in different wells. More specifically, the cell can be an Alternaria alternate, Aspergillus tumigatus, Candida glabrata, Cochiobolus heterostophus, Paracoccidioides brasiliensis, Pichia pastoris, Schizosaccharomyces pombe, Saccharomyces cerevisiae, Candida albicans, Aspergillus parasiticus, Ustalago maydis, Neurospora crassa, or Magnaporthe grisea. One or more of these cell types can be screened together if desired.

The screening methods can also be carried out in vitro, in which case one would prepare a cellular extract or a protein prep that includes a member of the Yap family (or a fragment or other variant thereof that retains a desired activity of the full-length Yap family member) or a gene encoding a Yap family member. The in vitro test can otherwise be carried out in the same manner as a cell-based test; the agent would be brought into contact with the protein or gene and one would then determine whether the agent could inhibit the protein's activity or the gene's expression.

The activity assayed can be oxidative stress; agents that inhibit the ability of the Yap family member to modulate oxidative stress are putative antifungal agents. To assess this ability one can, for example, place a reporter gene under the control of a regulatory region (e.g., the promoter) of a gene that is upregulated by Yap1 in the event of oxidative stress. The reporter gene can encode luciferase, GFP, or any other detectable product. In addition to assessing the ability of the test agent to mediate oxidative stress, one can assess the effect of the test agent on the ability of the Yap family member to regulate a cellular carrier, pump, or transporter. An agent that inhibits the ability of the protein (the Yap family member) to modulate oxidative stress and/or also inhibits the ability of the protein to regulate a cellular carrier, pump, or transporter of IAA is a putative antifungal agent. Blocking a cellular carrier, pump, or transporter of IAA alone may be sufficient, but agents that affect the ability of a Yap family member to mediate oxidative stress as well as inhibit IAA importation are likely to be superior to agents that affect only importation as there are numerous homologs of the transporter. The assay for transporter function can be carried out, for example, by providing a fungal cell with labeled IAA (e.g., radiolabeled IAA or IAA that is otherwise detectably labeled) and determining whether or not the labeled IAA is internalized by the cell.

To facilitate screening of numerous putative antifungal agents, the methods described herein can be configured as high throughput screens.

The present invention also features methods of screening for antifungal agents by identifying agents that inhibit IAA. These methods can be carried out by steps including: (a) providing an agent; (b) bringing the agent into contact with indole-3-acetic acid (IAA); and (c) determining whether the agent inhibits an activity of the IAA. Inhibition of an activity of IAA indicates that the agent is a putative antifungal agent.

As with the methods described above, virtually any agent can be screened, including small molecules (e.g., analogues of IAA), polypeptides, and nucleic acids, and the methods can be carried out using whole cells (including those listed above), cellular extracts, or any preparation containing IAA. At any stage, but likely after a first round of screening, the putative antifungal agent can be tested for its ability to inhibit fungal infection or filimentation in a tissue culture setting or in vivo.

Whether the methods are configured to assess IAA or a member of the Yap family, they can be carried out on varying scales, including high-throughput screens configured to assess hundreds or thousands of agents quickly.

Whether the methods are configured to assess IAA or a member of the Yap family, they can include further or additional steps carried out in animal models or clinical trials to assess the putative antifungal agent. For example, after an agent with the desired activity toward the target molecule (i.e., a Yap family member, including the protein of interest of the gene encoding it, or IAA) has been identified, the agent can be tested for its ability to inhibit the growth of fungi. This can be done, for example, by assessing the cell doubling time during, for example, the log phase of growth. For example, in rich media, wild-type C. albicans has a doubling time of approximately 60 minutes. Growth of the cells may be measured by following the optical density of the cells in liquid media, where an increasing optical density indicates growth. Alternatively, growth can also be measured by colony formation from single cells on solid media plates. One can also assess viability, or the ability of fungal cells to resume growth following a treatment of the cells that has resulted in cessation of growth. One way to assess viability is to test the ability of cells to form colonies on solid media plates.

Oligonucleotides that specifically bind and inhibit the expression of a gene encoding a Yap family member are within the scope of the present invention.

The details of one or more embodiments of the invention are set forth in the accompanying drawings and the description below. Other features, objects, and advantages of the invention will be apparent from the description and drawings, and from the claims.

### DETAILED DESCRIPTION

Many plant-associated microbes synthesize the auxin indole-3-acetic acid (IAA), and several IAA-biosynthetic pathways have been identified in microbes and plants. Saccharomyces cerevisiae has previously been shown to respond to IAA by inducing pseudohyphal growth. We observed that IAA also induced hyphal growth in the human pathogen Candida albicans, and thus may function as a secondary metabolite signal that regulates virulence traits such as hyphal transition in pathogenic fungi. Aldehyde dehydrogenase (ALD) is required for IAA synthesis from a tryptophan (Trp) precursor in Ustilago maydis. Mutant S. cerevisiae with deletions in two ALD genes are unable to convert radiolabeled Trp to IAA, yet produce IAA in the absence of exogenous Trp and at levels higher than wild type. These data suggest that yeast may have multiple pathways for IAA synthesis, one of which is not dependent on Trp.

IAA: The auxin indole-3-acetic acid (IAA) is best known for the role it plays in plant cell elongation, division and differentiation (Abel and Athanosios, “Odyssey of Auxin” in Cold Spring Harbor Perspectives in Biology, Cold Spring Harbor Laboratories Press, Cold Spring Harbor, N.Y., 2010; Halliday et al., “Integration of Light and Auxin Signaling” in Cold Spring Harbor Perspectives in Biology, Cold Spring Harbor Laboratories Press, Cold Spring Harbor, N.Y., 2009; McSteen, “Auxin and Monocot Development” in Cold Spring Harbor Perspectives in Biology, Cold Spring Harbor Laboratories Press, Cold Spring Harbor, N.Y., 2010; Moller and Weijers “Auxin Control of Embryo Patterning” in Cold Spring Harbor Perspectives in Biology, Cold Spring Harbor Laboratories Press, Cold Spring Harbor, N.Y., 2009; Scarpella et al., “Control of Leaf and Vein Development by Auxin” in Cold Spring Harbor Perspectives in Biology, Cold Spring Harbor Laboratories Press, Cold Spring Harbor, N.Y., 2010; Sundberg and Ostergaard, “Distinct and Dynamic Auxin Activities During Reproductive Development” in Cold Spring Harbor Perspectives in Biology, Cold Spring Harbor Laboratories Press, Cold Spring Harbor, N.Y., 2009; Zazimalova et al., “Auxin Transporters—Why So Many?” in Cold Spring Harbor Perspective in Biology, Cold Spring Harbor Laboratories Press, Cold Spring Harbor, N.Y., 2009). However, IAA has been identified in numerous plant-associated bacteria (reviewed in Glick et al., pp. 86-133 in Biochemical and Genetic Mechanisms Used by Plant Growth Promoting Bacteria, Glick et al., Eds., Imperial College Press, London, 1999) and several fungi, including Rhizopus suinous (Thimann, J. Biol. Chem. 109:279-291, 1935) Rhizoctonia (Furukawa et al., Plant Cell Physiol. 37:899-905, 1996), Colletotrichum (Robinson et al., Appl. Environ. Microbiol. 64:5030-5032, 1998), and yeast (Nielsen, Biochemische Zeitschrift 237:244-246, 1931; Gruen, Ann. Rev. Plant Physiol. 10:405-441, 1959). Microbial IAA plays a significant role in plant-microbe interactions (Glick et al., pp. 86-133 in Biochemical and Genetic Mechanisms Used by Plant Growth Promoting Bacteria, Glick et al., Eds., Imperial College Press, London, 1999), both pathogenic and symbiotic (Hirsch et al., Proc. Natl. Acad. Sci. USA 86:1244-1248, 1989; Reineke et al., Mol. Plant Pathol. 9:339-355, 2008).

Plants infected with pathogenic microbes manifest phenotypes consistent with elevated levels of IAA, such as gall formation (a tumor resulting from cellular proliferation) and lengthening of the stem (Barash and Manulis-Sasson, Phytopathol. 47:133-152, 2009; Stewart and Nemhauser, “Auxin as the Currency of the Cellular Economy” in Cold Spring Harbor Perspectives in Biology, Cold Spring Harbor Laboratories Press, Cold Spring Harbor, N.Y., 2009; Viglierchio, Botanical Rev. 37:1-21, 1971). The interplay between microbial-derived IAA and plant-derived IAA in plant disease is just beginning to be defined.

Exogenous IAA regulates filamentation in S. cerevisiae, a fungus that is primarily associated with plants, by inducing expression of genes that mediate it's morphological transition from vegetative form to a pseudohyphal or filamentous form (Prusty et al., Proc. Natl. Acad. Sci. USA 101:4153-4157, 2004). The fungal transcription factor, Yap1, regulates IAA homeostasis in S. cerevisiae (Prusty et al., Proc. Natl. Acad. Sci. USA 101:4153-4157, 2004) by down regulating auxin permeases (Avt proteins) that import IAA in S. cerevisiae (Prusty et al., Proc. Natl. Acad. Sci. USA 101:4153-4157, 2004). We show here that IAA stimulates filamentation in the human pathogen C. albicans, and the C. albicans Yap1 (Cap1) also mediates IAA phenotypes. Filamentation often underlies the development of virulence of C. albicans. For example, the C. albicans double mutant cph1Δ/Δ efg1Δ/Δ is defective in the MAP kinase pathway through Cph1, as well as the PKA pathway via Efg1. This mutant fails to switch from vegetative to filamentous form (Brown et al., Mol. Microbiol. 34:651-662, 1999; Liu, Curr. Opin. Microbiol. 4:728-735, 2001; Lo et al., Cell 90:939-949, 1997; Riggle et al., Infect. Immun. 67:3649-3652, 1999; Sohn et al., Mol. Microbiol. 47:89-102, 2003) and is also avirulent (Dieterich et al., Microbiol. 148:497-506, 2002). These studies suggest that the secondary metabolite IAA is a chemical signal that regulates fungal pathogenesis.

Plants have multiple pathways to synthesize, inactivate and catabolize IAA (Delker et al., Planta 227:929-941, 2008; Lau et al., Plant Cell 20:1738-1746, 2008; Normanly, Approaching Cellular and Molecular Resolution of Auxin Biosynthesis and Metabolism, Cold Spring Harbor Laboratories Press, Cold Spring Harbor, N.Y., 2009). Molecular genetic studies in model systems such as Arabidopsis thaliana (reviewed in Normanly, Approaching Cellular and Molecular Resolution of Auxin Biosynthesis and Metabolism, Cold Spring Harbor Laboratories Press, Cold Spring Harbor, N.Y., 2009), coupled with precise analytical methods (Barkawi et al., Anal. Biochem. 372:177-188, 2008) have helped expose some redundancy within this network. In fungi, IAA has been generally proposed as a metabolite of Trp (Hazelwood et al., Appl. Environ. Microbiol. 74:2259-2266, 2008) but this has been conclusively demonstrated only in U. maydis (Reneke et al., Cell 55:221-234, 1988) and S. uvarum (Shin et al., Chem. Pharm. Bull. (Tokyo) 39:1792-1795, 1991). Early studies used activity assays or qualitative colorimetric techniques to indicate the presence of IAA. Thin layer chromatography (TLC) and high performance liquid chromatography (HPLC) were subsequently employed for the detection of IAA, where the bioactive compound was shown to chromatograph with authentic IAA. Definitive isotope dilution quantification of IAA was first carried out with [¹⁴C]IAA and extracts from Ustilago zeae tumors (Turian and Hamilton, Biochim. Biophys. Acta. 41:148-150, 1960).

Agents that can be screened: Virtually any chemical or biological entity can be screened in the methods described herein. Agents useful as antifungal agents can be identified from libraries (e.g., combinatorial or compound libraries, including those that contain synthetic and/or natural products, and custom analog libraries, which may contain compounds based on a common scaffold). Such libraries can include hundreds or thousands of distinct compounds or random pools thereof. Libraries suitable for screening can be obtained from a variety of sources, including the compound libraries from ChemBridge Corp. (San Diego, Calif.). Another compound library is available from the consortium formed by the University of Kentucky, the University of Cincinnati Genome Research Institute and the Research Institute of the Children's Hospital of Cincinnati. The library is referred to as the UC/GRI Compound Library. The compound libraries employed in this invention may be prepared by methods known in the art. For example, one can prepare and screen compounds that target a YAP family member by any means including, but not limited to, combinatorial chemistry techniques, fermentation methods, plant and cellular extraction procedures and the like. Methods for making combinatorial libraries are well-known in the art. See, for example, E. R. Felder (Chimia 48:512-541, 1994); Gallop et al. (J. Med. Chem. 37:233-1251, 1994); R. A. Houghten (Trends Genet. 9:235-239, 1993); Houghten et al. (Nature 354:84-86, 1991); Lam et al. (Nature 354:82-84, 1991); Carell et al. (Chem. Biol. 3:171-183, 1995); Madden et al. (Perspectives in Drug Discovery and Design 2:269-282); Cwirla et al. (Biochemistry 87:6378-6382, 1990); Brenner et al. (Proc. Natl. Acad. Sci. USA 89:5381-5383, 1992); Gordon et al. (J. Med. Chem. 37:1385-1401, 1994); Lebl et al. (Biopolymers 37:177-198, 1995); and references cited therein.

The agents can also be screened from crude preparations of biological materials from plants, microorganisms, and animal sources. The agents can also be within partially purified extracts. In one embodiment, a crude preparation may be screened first and then further purified and tested if antifungal activity is detected in the crude extract.

Kits: In a further aspect, the invention relates to a kit for carrying out the in vitro screening method of the invention as first defined in the description. The kit comprises in separate containers (i) a polypeptide involved in cell wall synthesis, and (ii) a substrate for said polypeptide. It may further contain markers and controls. Preferably, the polypeptide present in the kit is any one of the preferred polypeptides defined earlier. Accordingly, in a further aspect, the invention relates to the use of this kit for performing the in vitro screening method as first described in the description.

### EXAMPLES

In the studies described below, we used gas-chromatography mass spectrometry (GC-MS) coupled with stable isotope dilution to demonstrate that S. cerevisiae synthesizes IAA. We identified genes homologous to the aldehyde dehydrogenase that functions in a Trp-dependent IAA biosynthetic pathway in U. maydis. Our results are consistent with the presence of a Trp-independent IAA biosynthetic pathway in yeast as well.

Strains, Media and Growth Conditions: Table 1 lists the strains used in this study. Deletion strains were derived from the yeast-deletion set (Winzeler et al., Science 285:901-906, 1999) and subsequently re-constructed by replacement of the relevant ORF with a dominant drug resistance marker (Wach et al., Yeast 10:1793-1808, 1994).

TABLE 1

| Strain | Description | Source |
|---|---|---|
| BY4741 | MATa; his3Δ1; leu2Δ0; met15Δ0; ura3Δ0 | YDL* |
| ald2Δ | ald2Δ in BY4741 | YDL |
| ald3Δ | ald3Δ in BY4741 | YDL |
| ald4Δ | ald4Δ in BY4741 | YDL |
| ald5Δ | ald5Δ in BY4741 | YDL |
| ald6Δ | ald6Δ in BY4741 | YDL |
| ald2Δ ald3Δ | ald2Δ ald3Δ in BY4741 | This study |
| Σ1278b | MATa; ura3Δ0 | Prof. Heitman, Duke Univ. |
| ald2Δ/Δ ald3Δ/Δ | ald2Δ ald30 in Σ1278b, mat a/α | This study |
| Caf2-1 | Candida albicans wild type | Prof. Fink, MIT |
| cph1Δ/Δ | Homozygous cph1Δ in Caf2-1 | Prof. Fink, MIT |
| efg1Δ/Δ | Homozygous efg1Δ in Caf2-1 | Prof. Fink, MIT |
| cph1Δ/Δ efglΔ/Δ | Homozygous cph1Δ efglΔ in Caf2-1 | Prof. Fink, MIT |
| cap1Δ/+ | Heterozygous cap1Δ in Caf2-1 | Prof. Raymond, U. Montreal |
| cap1Δ/Δ | Homozygous cap1Δ in Caf2-1 | Prof. Raymond, U. Montreal |

YDL*—Yeast Deletion Library

Analytical and phenotypic studies were performed in cognate deletion mutants, made in the Σ1278b background. A [¹⁴C]Trp incorporation assay was performed to verify that phenotype observed in the library strain could be recapitulated in the newly constructed E1278b strain. Typically three independent transformants were isolated, confirmed by PCR and used for further studies. Standard culture conditions were used (Sherman et al., Laboratory Course Manual for Methods in Yeast Genetics, Cold Spring Harbor Laboratories Press, Cold Spring Harbor, N.Y., 1986) and analysis of IAA-associated phenotypes were performed as described earlier (Prusty et al., Proc. Natl. Acad. Sci. USA 101:4153-4157, 2004).

[¹⁴C]Trp incorporation assay: Yeast strains were grown in 5 ml overnight cultures with aeration at 30° C. in Synthetic Complete medium (Sigma Inc.) (Guthrie and Fink, Methods in Enzymol. 194:______-______, 1991). To estimate cell density, the absorbance at 600 nm was measured and the culture was adjusted to an OD₆₀₀ of 1 (approximately 2×10⁷ cfu/ml). Cells (1 ml) were harvested by centrifugation at 3000 rpm for 5 minutes in an Eppendorf table-top microfuge at room temperature. Cells were washed twice by re-suspending pellets in water and then harvested by centrifugation. Cell pellets were re-suspended in 200 μl of SD medium supplemented with auxotrophic amino acids (Guthrie and Fink, Methods in Enzymol. 194, 1991). Samples were incubated with rocking (Thermolyne, speci mix) at 30° C. for approximately 18 hr in media containing 400 μM Trp and 0.5 μCi of [¹⁴C]Trp (Trp L-[side chain-3-¹⁴C], specific activity −50 mCi/m mol, American Radiochemicals Inc.). Cells were removed by centrifugation (3000 rpm in an Eppendorf table-top microfuge) at room temperature and the conditioned medium (CM) was transferred to new tubes for thin layer chromatography (TLC). Control samples were prepared identically but without the addition of cells to the SD medium. 10 μl of the CM was spotted on TLC plates. The [¹⁴C]Trp metabolites in the CM were resolved on a silica gel 60 F₂₅₄ (20×20 cm, 250 μM thick precoated) TLC plate (EMD Chemicals Inc.). A mixture of 85% chloroform, 14% methanol, and 1% water was used as the eluting solvent. IAA that had incorporated label from [¹⁴C]Trp was visualized by autoradiography. Commercially available [¹⁴C]IAA (American Radiochemicals Inc.) was used as a standard. In order to screen the yeast deletion set, this assay was adapted for use in 96-well microtiter dishes by scaling down the reaction volume to 50 μl containing 0.1 μCi [¹⁴C]Trp.

Quantification of IAA from yeast: To confirm that IAA was present in the CM, 5 mL cultures, were harvested and stored at −80° C. The supernatants were thawed on ice and 38.4 ng of [¹³C₆]IAA (99 atom %, Cambridge Isotope Laboratories) in 10 μL of 2-propanol were added as an internal standard. Additionally, 500 pμL of 0.2 M imidazole (pH 7.0) were added. The sample was mixed and left to equilibrate on ice for 1 hr. The sample was loaded onto a 200 mg NH₂ solid phase extraction (SPE) column (aka amino columns, Alltech) that was pre-conditioned with sequential applications of 2 mL each hexane, acetonitrile, water, and 0.2 M imidazole (pH 7.0) followed by 6 mL of water on a vacuum manifold (Fisher Scientific). After loading the sample, the column was allowed to aspirate under vacuum for an additional 30 sec at 3-5 psi. Next, the column was washed with sequential additions of 1 mL each of hexane, ethyl acetate, acetonitrile and methanol. IAA was eluted in approximately 6 mL of methanol that was 5% acetic acid. Dried samples were resuspended in 1.3 mL of a mixture (approximately 6:1 to reach a pH between 3-3.5) of 0.25% phosphoric acid and 0.1 M succinic acid, pH 6.0. The sample was placed in a 2 mL capacity 96-well plate and subjected to an additional SPE step with polymethymethacrylate epoxide resin using a Gilson 215 SPE automated liquid handler (ALH) as described in Barkawi et al. (Anal. Biochem. 372:177-188, 2008). The epoxide SPE column eluate was transferred to 2 mL amber vials, and approximately 1 mL of ethereal diazomethane (prepared as described in Cohen, J. Chromatog. 303:193-196, 1984) was added. After a 5 minute incubation at room temperature, the sample was dried to a residue under a stream of N₂ gas in a 45° C. sand bath. The methylated IAA was resuspended in 45 μL of ethyl acetate and subjected to GC-MS analysis as described in Barkawi et al. (Anal. Biochem. 372:177-188, 2008), except that a full scan spectrum was obtained. For mutant analysis, this protocol was scaled down to 1 ml cultures containing the same amount of [¹³C₆]IAA internal standard but only 0.2 mL of 0.2 M imidazole, pH 7.0.

S. cerevisiae secretes IAA: To confirm that S. cerevisiae synthesizes IAA, we analyzed conditioned medium (CM) from yeast cultures. Thin layer chromatography of CM from S. cerevisiae grown in the presence of [¹⁴C]Trp revealed a radiolabeled product that co-migrated with commercially available [¹⁴C]IAA. UV-shadow of the fluor-impregnated TLC plate showed a UV absorbing compound with the same retention profile as the pure unlabeled IAA that was used as a standard. GC-MS analysis of IAA that was extracted from the CM along with [¹³C₆]IAA internal standard and methylated for GC analysis confirmed the presence of IAA in the CM. The total ion chromatogram (TIC) of pure methyl (Me)-IAA showed a GC retention time for authentic Me-IAA to be between 7.322 to 7.380 min. The predominant ions for pure Me-IAA are m/z 189 (intact Me-IAA, aka molecular ion), and m/z 130 (fragment ion). The observed fragment ions of m/z 103 and 77 were consistent with pure Me-IAA as well, but were lower in abundance and not typically used for quantification. Our results demonstrate that yeast secretes IAA.

The accumulation of IAA in the CM reached its highest level after cultures entered stationary phase. To correlate the production of IAA with cell density, cells from a high-density culture (10⁸ cells/mL) were diluted to either low (5×10⁵ cells/mL) or high density (5×10⁷ cells/mL) in fresh medium. IAA secreted into the medium was assessed by TLC. After normalizing for the difference in cell number, we found that CM taken from a high-density culture contained more IAA than CM from a low-density culture, indicating that IAA accumulation is directly proportional to cell density. In S. cerevisiae, IAA is perceived, imported and stimulates diploid pseudohyphal growth and haploid invasive growth by regulating the cell surface glycoprotein Flo 11. Together these studies suggest that IAA accumulates in the growth environment of yeast where it may acts as a chemical signal that regulates virulence traits.

A genomic scale screen for IAA homeostasis mutants: To identify genes involved in IAA synthesis, specifically, the conversion of Trp to IAA, we initiated an unbiased, systematic genomic screen of the yeast deletion library (Brachmann et al., Yeast 14:115-132, 1998; Winzeler et al., Science 285:901-906, 1999). The haploid deletion library in S. cerevisiae consists of approximately 4940 clones representing every single viable gene disruption. A [¹⁴C]Trp incorporation assay was developed and optimized to facilitate a large-scale screen using microtiter plates. An aliquot of the CM from each reaction was loaded onto a TLC plate and components of the CM were resolved and compared with ¹⁴C-IAA standard. A total of 1425 deletion strains (29% of the library) have been screened to date. A secondary screen was performed in triplicate on putative mutants and related gene families using the [¹⁴C]Trp incorporation assay.

This screen identified three genes, ALD2, ARO9, and ADH2, representing families of particular interest with respect to IAA biosynthesis in yeast: the ALDehyde dehydrogenases, the AROmatic transaminases and the Alcohol DeHydrogenases. In S. cerevisiae, the aromatic transaminases, Aro8 and Aro9 have been implicated in the conversion of Trp to indole pyruvate (IPA) (Chen and Fink, Genes Dev. 20:1150-1161, 2006). As expected, aro8Δ and aro9Δ mutants show decreased conversion of labeled Trp to labeled IAA compared to the cognate wild type but are not the focus of this study. Alcohol dehydrogenases are proposed to convert indole acetaldehyde (IAA1d) to indole-3-ethanol (aka tryptophol) (Chen and Fink, Genes Dev. 20:1150-1161, 2006). Interestingly, adh2Δ, identified in the screen, was the only member of the ADH family to show decreased [¹⁴C]IAA accumulation. One explanation for this result is that Adh2 preferentially catalyzes the conversion of ethanol to acetylaldehyde. Therefore adh2Δ mutants are unable to convert indole-3-ethanol to IAA1d, ultimately leading to decreased IAA accumulation. Deletion mutants of members of the ALD family accumulated lower levels of radioactive IAA from radioactive Trp than did wild type. We focused our study on the aldehyde dehydrogenase (ALD) genes hypothesized to catalyze the ultimate step in the production of IAA and set out to test whether altering IAA production affects filamentation. Multiple sequence alignment and phylogenetic analysis indicates that S. cerevisiae Ald2 and Ald3 share identity with U. maydis Iad1. Ald2 and Ald3 are nearly identical to each other and have 50% (Ald3) and 49% (Ald2) protein sequence identity with U. maydis Iad1, a NAD-dependent aldehyde dehydrogenase. Ald2 and Ald3 have less sequence identity with the NADH-dependent aldehyde dehydrogenases such as lez O. Single and double deletions of the ALD genes showed decreased IAA production from [¹⁴C]Trp when compared with wild-type cells on TLC. These results together with previous enzymatic studies in U. maydis (Reineke et al., Mol. Plant Pathol. 9:339-355, 2008) suggest that these genes are involved in IAA synthesis. ALD2 and ALD3 are also required for synthesis of a non-proteinogenic amino acid, β-alanine in S. cerevisiae (White et al., Genetics 163:69-77, 2003).

The ald2Δald3Δ deletion mutant exhibits virulence traits: IAA regulates dimorphic transition in S. cerevisiae by inducing adhesion and filamentation (Prusty et al., Proc. Natl. Acad. Sci. USA 101:4153-4157, 2004). The ability of a fungus to perceive a small molecule signal that causes it to differentiate into an invasive form has important implications for host-pathogen interactions. To test the hypothesis that mutants with aberrant IAA accumulation also affect dimorphism, we examined diploid filamentation and haploid invasive growth in all ald single mutants and selected combinations of double mutants. The ald2Δald3Δ double mutant demonstrated increased filamentation and invasive growth as compared wild type. We also tested a previously reported growth inhibition phenotype associated with exposure to IAA. This IAA-associated growth inhibition phenotype exhibits a direct proportionality between IAA concentration and growth inhibition. Deletion of both ald2 and ald3 caused an increase in sensitivity to IAA whereas single deletion of an ALD gene did not affect IAA sensitivity when compared with wild-type cells. Together, these data suggest that a perturbation in the IAA secretion profile alters substrate adhesion and filamentation of S. cerevisiae. However, these phenotypes are consistent with ald2Δald3Δ mutants producing more IAA than isogenic wild-type strains.

The ald2Δald3Δ mutant uncovers an IAA biosynthetic pathway that is independent of exogenous Trp: The ALD genes were identified based on a radiolabeled [¹⁴C]Trp incorporation assay. IAA accumulation in the CM of the double mutant was quantified using GC-MS and [¹³C₆]IAA as an internal standard. These measurements revealed that the CM of the ald2Δald3Δ deletion mutant contained four-fold more IAA (240.3 ng/mL +/−71.9 ng/mL) than the wild type (59.8 ng/mL +/−3.8 ng/mL). The amount of IAA present in the conditioned media is adequate to induce filamentation in an in vitro plate assay. Together these analytical data correlate well with the phenotypic data, suggesting that the ald2Δald3Δ double mutant makes more IAA and thus exhibits enhanced virulence traits as compared to its wild-type counterpart.

While the radiolabeled [¹⁴C]Trp incorporation assay detects the pool of IAA synthesized from labeled Trp, the GC-MS analysis allowed us to detect any unlabeled (endogenous) IAA that was present. We grew the ald2Δald3Δ double mutant in the absence of exogenous Trp and quantified IAA from the CM using GC-MS. These measurements revealed that the ald2Δald3Δ mutant was able to synthesize a modest amount of IAA (9.48 ng/mL +/−0.22 ng/mL) in the absence of exogenous Trp. Wild type yeast also produced similar amount of IAA in the absence of Trp (9.81 ng/mL +/−0.77 ng/mL).

IAA induces filamentation in C. albicans: The effects of the secondary metabolites identified in fungi appear to be largely species specific (Chen and Fink, Genes Dev. 20:1150-1161, 2006). Previous work suggests that IAA induces invasive growth in S. cerevisiae Prusty et al., Proc. Natl. Acad. Sci. USA 101:4153-4157, 2004). To test whether the IAA effects could cross species barriers, we exposed wild-type C. albicans, a human pathogen, as well as attenuated mutants in the mitogen-activated protein (MAP) kinase and the cAMP-dependent protein kinase pathways to IAA. The cph1Δ/Δefg1Δ/Δ double mutant, which fails to switch from vegetative to the filamentous form, was filamentous in the presence of IAA. The single mutants, efg1Δ/Δ or cph1Δ/Δ that normally show reduced filamentation also showed a robust filamentation when exposed to IAA. Wild-type strains also filamented more when treated with IAA as compared to untreated cells. These results indicate that IAA enhances filamentation of the human pathogen C. albicans. Furthermore, the IAA-mediated filamentation signal does not require components of the MAPK or PKA pathways. The cph1Δ/Δefg1Δ/Δ double mutant, which is nonfilamentous under standard laboratory conditions and avirulent in mice, filaments in the oral cavity of immunosuppressed piglets and when embedded in agar (Riggle et al., Infect. Immun. 67:3649-3652, 1999). Together these results suggest that IAA mediated filamentation in C. albicans occurs via an Efg1p and Cph1p-independent mechanism and confirms prior findings of the existence of an alternate filamentation pathway in C. albicans.

To test if other aspects of IAA regulation in S. cerevisiae were also conserved in C. albicans, we tested Cap1, the C. albicans homolog of Yap, for its sensitivity to IAA. The Amino Acid Auxin Permeases genes are upregulated in the yap1 mutant, which is sensitive to growth on IAA because it retains more IAA (Prusty et al., Proc. Natl. Acad. Sci. USA 101:4153-4157, 2004). Heterozygous and homozygous deletion mutants of CAP1 (Alarco and Raymond, J. Bacteriol. 181:700-1708, 1999) (obtained from Dr. M. Raymond, University of Montreal) to grew less well on media containing IAA as compared to the isogenic wild type, suggesting that the cap1Δ/Δ mutant was more sensitive to IAA. The heterozygous mutant, cap1Δ/+ exhibited an intermediate sensitivity to IAA as compared to the wild-type CAP1+/+strain or the homozygous cap1Δ/Δ deletion strain. These results suggest that cap1 mutants are hypersensitive to IAA, further supporting our hypothesis that the molecular mechanism of IAA response is likely to be conserved between S. cerevisiae and C. albicans.

The quantitative GC-MS analysis in the study described above confirmed that S. cerevisiae synthesizes and secretes IAA into the culture environment where it is available to function as a signal that regulates filamentation. Filamentation is a pathogenic trait because it contributes directly to virulence of pathogenic fungi like C. albicans. Pathogenic bacteria and fungi are known to produce IAA, but a direct link to pathogenicity has not been demonstrated in these pathogens.

IAA is a small molecule capable of stimulating the developmental transition from the vegetative yeast form to filamentous form in S. cerevisiae (Prusty et al., Proc. Natl. Acad. Sci. USA 101:4153-4157, 2004). The current study provides strong support for a connection between fungal dimorphism and IAA synthesis, because the ald2Δald3Δ strain that accumulates more IAA is also more filamentous. IAA was also able to stimulate dimorphic transition in the human pathogen C. albicans. Deletion of a key regulator of the IAA responses had the same effect in both organisms. Homologs of enzymes that transport and synthesize IAA S. cerevisae are present in C. albicans. We suggest that IAA is an important signal that triggers dimorphic transition—a virulence trait.

A genomic scale screen for IAA homeostasis mutants implicated the aldehyde dehydrogenases, Ald2 and Ald3 in the final step of IAA synthesis from Trp. Ald2 and Ald3 share significant sequence similarity with Iad1, the U. maydis aldehyde dehydrogenase that has been shown to catalyze the conversion of IAA1d to IAA (Akamatsu et al., J. Biosci. Bioeng. 90:555-560, 2000; Basse et al., Eur. J. Biochem. 242:648-656, 1996; Mizuno et al., J. Biosci Bioeng. 101:31-37, 2006; Pigeau and Inglis, J. Appl. Microbiol. 103:1576-1586, 2007; Reineke et al., Mol. Plant Pathol. 9:339-355, 2008). The ALD genes are responsible for acetate formation during anaerobic fermentation (Pigeau and Inglis, J. Appl. Microbiol. 103:1576-1586, 2007; Saint-Prix et al., Microbiol. 150:2209-2220, 2004), hence of interest to the brewing industry. They have previously been implicated in mediating a variety of stress responses and are regulated by general-stress transcription factors Msn2,4 (Aranda and del Olmo, Yeast 20:747-759, 2003; Miralles and Serrano, Mol. Microbiol. 17:653-662, 1995; Navarro-Avino et al., Yeast 15:829-842, 1999). Ald activity is required in the synthesis of two amino acid derivatives, IAA and β-alanine in U. maydis and S. cerevisiae respectively (Reineke et al., Mol. Plant Pathol. 9:339-355, 2008; White et al., Genetics 163:69-77, 2003). This screen identified other members of the pathway (Aro9), which has previously been implicated in the first step of IAA synthesis (Chen and Fink, Genes Dev. 20:1150-1161, 2006). In the process of characterizing mutants in a Trp-dependent IAA synthesis pathway, we uncovered another pathway that did not rely on exogenous Trp for IAA biosynthesis. Trp-independent synthesis of IAA has been demonstrated in several plant species, but the intermediates, intermediate steps, and genes involved in this pathway remain undefined (Normanly, Approaching Cellular and Molecular Resolution of Auxin Biosynthesis and Metabolism, Cold Spring Harbor Laboratories Press, Cold Spring Harbor, N.Y., 2009; Woodward and Bartel, Ann. Bot. (London) 95:707-735, 2005). The observation that S. cerevisiae has an analogous pathway provides a much simpler system to employ in the characterization of Trp-independent IAA synthesis.

There is precedence for multiple IAA biosynthetic pathways in microbes, particularly plant-associated bacteria (Costacurta and Vanderleyden, Crit. Rev. Microbiol. 21:1-18, 1995; Glick et al., pp. 405-441 in Biochemical and Genetic Mechanisms Used by Plant Growth Promoting Bacteria, Glick et al., Eds., Imperial College Press, London, 1999; Clark et al., Mol. Plant Pathol. 83:234-240, 1993; Lambrecht et al., Trends Microbiol. 8:298-300, 2000). An interesting example of differential utilization of multiple IAA biosynthetic pathways in microbes is found in Erwinia herbicola, which requires a functional indole acetamide (JAM) pathway (Trp is converted to IAM, then IAA) in order to be pathogenic to plants and requires a functional IPA pathway to exist as a plant epiphyte (Manulis et al., Mol. Plant Microbe Interact. 11:634-642, 1998). We note that while aldehyde dehydrogenase has been implicated in IAA synthesis in U. maydis this pathway is not involved in tumorogenesis (Reineke et al., Mol. Plant Pathol. 9:339-355, 2008). This result is consistent with our observation that ALD2 and ALD3 are not necessary for IAA-induced filamentation and that an alternate IAA synthesis pathway likely exists in yeast.

The co-existence of both Trp-dependent and Trp-independent IAA-biosynthetic pathways has been documented in plants (Woodward and Bartel, Ann. Bot. (London) 95:707-735, 2005; Normanly et al., Auxin Metabolism in Plant Hormones: Biosynthesis, Signal Transduction, Action . . . Kluwer Academic Publishers, Dordrecht, The Netherlands, 2004) and microbes (Prinsen et al, Mol. Plant-Microbe Interact. 6:609-615, 1993). In plants, Trp-independent IAA synthesis is proposed to branch from either indole or indole glycerol phosphate, both precursors of Trp (Normanly et al., Auxin Metabolism in Plant Hormones: Biosynthesis, Signal Transduction, Action . . . Kluwer Academic Publishers, Dordrecht, The Netherlands, 2004). One of the proposed Tip-dependent IAA biosynthetic pathways for plants converts Trp to IPA (reviewed in (Normanly, Approaching Cellular and Molecular Resolution ofAuxin Biosynthesis and Metabolism, Cold Spring Harbor Laboratories Press, Cold Spring Harbor, N.Y., 2009; Woodward and Bartel, Ann. Bot. (London) 95:707-735, 2005). The Arabidopsis TAA-1 protein can convert Trp to IPA in vitro, and mutations in the TAA-1 gene produce less IAA when the plant is subjected to simulated shade (Tao et al., Cell 133:164-176, 2008), high temperature (Yamada et al., Plant Physiol. XX:xx-xx, 2009) or ethylene (Stepanova et al., Cell 133:177-191, 2008). IAA1d has been proposed as an intermediate of the Tip-dependent IAA synthetic pathway in plants, but this has yet to be confirmed, and plant orthologs of ALD genes have not been identified. One putative aldehyde oxidase from Arabidopsis shows a substrate preference for IAA1d in vitro, but the relevance of this gene to IAA biosynthesis in vivo has yet to be confirmed (Seo et al., Plant Physiol. 116:687-693, 1998). Future studies will involve using differential stable isotope labeling coupled with genetic mutants to identify components of alternate IAA biosynthetic pathways in S. cerevisiae.

Secondary metabolites are recognized as important signals. Aspergillus fumigatus hyphae release a small molecule, gliotoxin, which can exacerbate the pathogenesis of invasive aspergillosis (Sutton et al., Immunol. Cell Biol. 74:318-322, 1996). Pseudomonas aeruginosa produces a signaling molecule, homoserine lactone, which inhibits C. albicans filamentation (Hogan et al., Mol. Microbiol. 54:1212-1223, 2004). Two predominant types of small-molecules, acyl homoserine lactones (AHLs) (Akimkina et al., FEMS Microbiol. Lett 264:145-151, 2006; Danhorn et al., J. Bacteriol. 186:4492-4501, 2004; Fuqua et al., Ann. Rev. Genet. 35:439-468, 2001) and modified oligopeptides (Kleerebezem et al., Mol. Microbiol. 24:895-904, 1997) are used by Gram-negative and Gram-positive bacteria, respectively, to regulate phenotypes that lead to virulence such as antibiotic production and biofilm formation. C. albicans has been shown to produce secondary metabolites such as tyrosol and farnesol that regulate dimorphic transition (Chen et al., Proc. Natl. Acad. Sci. USA 101:5048-5052, 2004; Shchepin et al., Chem. Biol. 10:743-750, 2003). Aromatic alcohols such as tryptophol and phenylalanol, a catabolic product of Phe are produced by both S. cerevisiae and C. albicans but exert different effects on their morphogenesis suggesting that they have distinct species-specific effects. IAA differs from these previously described signaling molecules because its effects appear to cross species barriers. Diverse fungal species respond to IAA, therefore, defining the pathways by which IAA regulates filamentation in C. albicans will yield a better understanding of its pathogenesis and potentially the development of broad-spectrum antifungal therapies. Furthermore, auxin permeases that import IAA in S. cerevisiae are homologous to the Arabidopsis IAA importer, Aux1 (Prusty et al., Proc. Natl. Acad. Sci. USA 101:4153-4157, 2004). Therefore, defining IAA synthesis and regulation in yeast, a simple eukaryote, will yield a better understanding of IAA regulation in plants.

A number of embodiments of the invention have been described. Nevertheless, it will be understood that various modifications may be made without departing from the spirit and scope of the invention. Accordingly, other embodiments are within the scope of the following claims.

What is claimed is:

1. A method of screening for an antifungal agent, the method comprising:
(a) providing an agent;
(b) bringing the agent into contact with a protein of the YAP family; and
(c) determining whether the agent inhibits an activity of the protein, wherein inhibition of an activity of the protein indicates that the agent is a putative antifungal agent.

2. The method of claim 1, wherein the agent is a small molecule, polypeptide, or nucleic acid.

3. The method of claim 1, wherein the protein is AaAP1, Afyap1, CgAP1, ChAP1, Yap1, Yap2, PpYap1, Pap1, Cap1, ApyapA, or Nap1 or a biologically active variant thereof

4. The method of claim 1, wherein bringing the agent into contact with the protein comprises bringing the agent into contact with a fungal cell that expresses the protein.

5. The method of claim 4, wherein the fungal cell is a cell of the genus Alternaria, Aspergillus, Candida, Cochiobolus, Paracoccidioides, Pichia, Schizosaccharomyces, Saccharomyces, Ustilago, Neurospora, Magnaporthe, or a combination thereof.

6. The method of claim 5, wherein the fungal cell is Alternaria alternate, Aspergillus tumigatus, Candida glabrata, Cochiobolus heterostophus, Paracoccidioides brasiliensis, Pichia pastoris, Schizosaccharomyces pombe, Saccharomyces cerevisiae, Candida albicans, Aspergillus parasiticus, Ustalago maydis, Neurospora crassa, Magnaporthe grisea, or a combination thereof

7. The method of claim 1, wherein the activity is modulation of oxidative stress.

8. The method of claim 8, wherein the agent inhibits the ability of the protein to modulate oxidative stress but does not inhibit the ability of the protein to regulate a cellular carrier, pump, or transporter.

9. The method of claim 1, wherein the steps are configured as a high throughput screen.

10. A method of screening for an antifungal agent, the method comprising:
(a) providing an agent;
(b) bringing the agent into contact with indole-3-acetic acid (IAA); and
(c) determining whether the agent inhibits an activity of the IAA, wherein inhibition of an activity of the IAA indicates that the agent is a putative antifungal agent.

11. The method of claim 10, wherein the agent is a small molecule, polypeptide, or nucleic acid.

12. The method of claim 10, wherein bringing the agent into contact with the IAA comprises bringing the agent into contact with IAA in a fungal cell or in the local environment of the fungal cell.

13. The method of claim 12, wherein the fungal cell is a cell of the genus Alternaria, Aspergillus, Candida, Cochiobolus, Paracoccidioides, Pichia, Schizosaccharomyces, Saccharomyces, Ustilago, Neurospora, Magnaporthe, or a combination thereof.

14. The method of claim 13, wherein the fungal cell is Alternaria alternate, Aspergillus tumigatus, Candida glabrata, Cochiobolus heterostophus, Paracoccidioides brasiliensis, Pichia pastoris, Schizosaccharomyces pombe, Saccharomyces cerevisiae, Candida albicans, Aspergillus parasiticus, Ustalago maydis, Neurospora crassa, Magnaporthe grisea, or a combination thereof.

15. The method of claim 10, wherein the steps are configured as a high throughput screen.
